(19) United States
(12) Patent Application Publication
(10) Pub. No.: US 2004/0170575 A1
(43) Pub. Date: Sep. 2, 2004

(54) Aerosol composition comprising silicate particles and polymers

(76) Inventors: Emmanuelle Belli, Asnieres (FR); Francoise Pataut, Paris (FR); Cecile Nocerino, Paris (FR); Audrey Gilles, Velizy (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/479,170

(22) PCT Filed: May 31, 2002
(86) PCT No.: PCT/FR02/01847

(30) Foreign Application Priority Data
May 31, 2001 (FR) 01/07156

(51) Int. Cl. A61K 7/06; A61K 7/11; A61K 9/00

(52) U.S. Cl. 424/47; 424/70.12

(57) ABSTRACT

The invention concerns a cosmetic composition, in particular for the hair, packaged in an aerosol device containing a propellant and a liquid phase which comprises, in a cosmetically acceptable medium: (i) solid particles containing at least 10 wt. % of at least a silicate, and (ii) at least a polymer selected among fixing polymers, thickening polymers or a mixture thereof; said solid particles contain at least 1 wt. % of aluminium.

The present invention relates to a composition packaged in an aerosol device comprising a propellant and a liquid phase which contains, in a cosmetically acceptable medium, silicate particles, to a cosmetic hair treatment process and to a use of this composition as a hairstyling product.

Hairstyling products are usually used to construct and structure the hairstyle and give it lasting hold. For this, filmogenic polymers are introduced into a cosmetically acceptable medium. However, certain polymers cause hardening of the head of hair and/or a tacky character. For this reason, the hair is often glued together and untangling causes destruction of the form of the head of hair.

To remedy these disadvantages, it has already been proposed to use solid powder in the cosmetic compositions, such as powders of metal oxides. For example, U.S. Pat. No. 3,819,827 describes products for setting hair comprising 0.2 to 6% by weight of aluminum oxide particles exhibiting a particle size of about 30 μm.

In the same way, patent application EP 1 005 849 describes the use of fine inorganic solid powders, such as the powders of calcium carbonate, aluminum hydroxide, magnesium carbonate, calcium phosphate, calcium oxalate or barium sulfate, in cosmetic hair compositions containing at least two liquid phases. It is specified that such compositions give the hair especially advantageous effects when the composition is packaged, with no propellant gas, in a device which can spray the composition onto the hair.

Patent DE 25 42 338 describes lotions and sprays, with no propellant, for fixing hair comprising silicate particles, in an alcohol or hydroalcohol medium, the concentration of silicates being between 0.1 and 4%.

The Applicant has found, quite surprisingly and unexpectedly, that by selecting, as solid particles, silicate particles, it is possible to obtain good cosmetic properties and to obtain a hairstyle without gluing or overloading the hair, insofar as the composition is packaged in a particular device, that is an aerosol device, when the cosmetic composition comprises, moreover at least one polymer selected from fixing polymers, thickening polymers or a mixture thereof.

The object of the invention is a cosmetic composition, notably for the hair, packaged in an aerosol device, containing a propellant and a liquid phase which comprises, in a cosmetically acceptable medium:

(i) solid particles containing at least 10% by weight of at least one silicate, and

(ii) at least one polymer selected from fixing polymers, thickening polymers or a mixture thereof;

said solid particles containing less than 1% by weight of aluminum.

Another object of the present invention consists of a cosmetic hair treatment process using the composition according to the invention.

The object of the invention is also the use of the composition in hair cosmetics, notably for fixing and/or maintaining the hair in a desired form.

Other objects, characteristics, aspects and advantages of the invention will appear even more clearly on reading the specification and the various examples which follow.

Particles

Preferably, the solid particles contain between 0 and 1% aluminum, and more preferentially still between 0 and 0.5%.

Advantageously, the solid particles have a number-average primary size of between 2 nm and 1 μm.

The particles containing at least 10% by weight of at least one silicate have, preferably, a number-average primary size of between 5 and 500 nm, and more preferentially between 10 and 250 nm.

The particles according to the invention can have any form, for example the form of spheres, flakes, needles, chips or completely random shapes.

Preferably, the silicates are those of sodium, magnesium and/or lithium, such as the compounds marketed by the Company Laporte under the names LAPONITE XLG and LAPONITE XLS.

Within the meaning of the present invention, by “primary particle size” is understood the maximum dimension that it is possible to measure between two diametrically opposite points of an individual particle. The size can be determined, for example, by transmission electron microscopy or by measuring the specific surface area by the BET method or by means of a laser particle size analyzer.

Where the particles are formed by silicate and other fillers, the silicate is in the free state and does not form chemical bonds with the other fillers. There is therefore an alloy between the silicate and other fillers, notably with metal or metalloid oxides, in particular obtained by thermal fusion of these different constituents.

When the particles containing at least 10% by weight of at least one silicate comprise, moreover, a metal or metalloid oxide, this is notably selected from silicon, boron and aluminum oxide.

Preferably, the particles contain at least 50% by weight of silicate, better still at least 70% by weight, and the particles constituted to more than 90% by weight of silicate are particularly preferred according to the present invention.

The suitable silicate in the compositions of the present invention can be of natural origin or can be of synthetic origin.

The particles containing silicate according to the invention are, notably, used in a quantity of between 0.01% and 10% by weight, and preferably between 0.5% and 3% by weight, in relation to the total weight of the composition.

The composition according to the invention can also contain other types of particles, for example, particles of titanium, zinc or aluminum oxide.

Fixing Polymers

The fixing polymer (b) is selected from cationic, anionic, amphoteric and nonionic fixing polymers and their mixtures. A fixing polymer is a polymer capable of maintaining and/or fixing the form of the hairstyle.

These fixing polymers can be used in solubilized form or even in the form of a dispersion of solid polymer particles.

The cationic, anionic, amphoteric and nonionic fixing polymers (b) which can be used in accordance with the invention are described below.

The anionic fixing polymers generally used are polymers comprising groups derived from carboxylic, sulfonic or phosphoric acid and have a molecular weight of between about 500 and 5,000,000.

1) The carboxylic groups are provided by unsaturated carboxylic mono- or diacid monomers such as those corresponding to the formula:

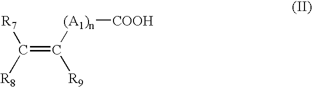

in which n is a whole number from 0 to 10, A₁ denotes a methylene group, possibly linked to the carbon atom of the unsaturated group or to the neighboring methylene group when n is greater than 1 by means of a heteroatom such as oxygen or sulfur, R₇ denotes a hydrogen atom, a phenyl or benzyl group, R₈ denotes a hydrogen atom, a lower alkyl or carboxyl group, R₉ denotes a hydrogen atom, a lower alkyl group, a —CH₂—COOH, phenyl or benzyl group;

In the above formula, a lower alkyl radical denotes preferably a group having 1 to 4 carbon atoms and in particular, methyl and ethyl.

The preferred anionic fixing polymers with carboxylic groups according to the invention are:

A) The homo- or copolymers of acrylic or methacrylic acid or their salts and in particular the products sold under the names VERSICOL E or K by the company ALLIED COLLOID and ULTRAHOLD by the company BASF. The copolymers of acrylic acid and acrylamide sold in the form of their sodium salt under the names RETEN 421, 423 or 425 by the Company HERCULES, the sodium salts of polyhydroxycarboxylic acids.

B) The copolymers of acrylic or methacrylic acids with a monoethylene monomer such as ethylene, styrene, vinyl esters, acrylic or methacrylic acid esters, possibly grafted on a polyalkylene glycol such as polyethylene glycol and possibly crosslinked. Such polymers are described in particular in French patent 1.222.944 and German application 2.330.956, the copolymers of this type comprising in their chain an acrylamide unit possibly N-alkylated and/or hydroxyalkylated such as described notably in Luxembourg patent applications 75370 and 75371 or proposed under the name QUADRAMER by the Company AMERICAN CYANAMID. The copolymers of acrylic acid and C₁-C₄ alkyl methacrylate and the terpolymers of vinylpyrrolidone, acrylic acid and C₁-C₂₀ alkyl methacrylate for example lauryl methacrylate such as that sold by the company ISP under the name ACRYLIDONE LM and the methacrylic acid/ethyl acrylate/tert-butyl acrylate terpolymers such as the product sold under the name LUVIMER 100 P by the company BASF, can also be cited.

C) the copolymers derived from crotonic acid such as those comprising in their chain vinyl propionate or acetate units and possibly other monomers such as allyl or methallyl esters, vinyl ether or vinyl ester of a saturated linear or branched carboxylic acid with a long hydrocarbon chain such as those comprising at least 5 carbon atoms, these polymers being able possibly to be grafted and crosslinked or even a vinyl, allyl or methallyl ester of an α- or β-cyclic carboxylic acid. Such polymers are described amongst others in French patents 1,222,944, 1,580,545, 2,265,782, 2,265,781, 1,564,110 and 2,439,798.

Commercial products included in this class are the resins 28-29-30, 26-13-14 and 28-13-10 sold by the company NATIONAL STARCH.

D) the copolymers derived from C₄-C₈ monounsaturated carboxylic anhydrides or acids selected from:

the copolymers comprising (i) one or several maleic, fumaric, itaconic acids or anhydrides and (ii) at least one monomer selected from vinyl esters, vinyl ethers, vinyl halides, phenylvinyl derivatives, acrylic acid and its esters, the anhydride functions of these copolymers being possibly monoesterified or monoamidified; Such polymers are described in particular in U.S. Pat. Nos. 2,047,398, 2,723,248, 2,102,113, patent GB 839,805 and notably those sold under the names GANTREZ AN or ES by the company ISP.

the copolymers comprising (i) one or several maleic, citraconic, itaconic anhydrides and (ii) one or several monomers selected from the allyl or methallyl esters comprising possibly one or several acrylamide, methacrylamide, a-olefin groups, acrylic or methacrylic esters, acrylic or methacrylic acids or vinylpyrrolidone in their chain,

the anhydride functions of these copolymers being possibly monoesterified or monoamidified.

These polymers are for example described in the applicant's French patents 2,350,384 and 2,357,241.

E) the polyacrylamides comprising carboxylate groups.

The polymers comprising the sulfonic groups are polymers comprising vinylsulfonic, styrenesulfonic, naphthalenesulfonic or acrylamidoalkylsulfonic units.

These polymers can be notably selected from:

the salts of polyvinylsulfonic acid having a molecular weight of between about 1,000 and 100,000 and copolymers with an unsaturated comonomer such as acrylic or methacrylic acids and their esters and acrylamide or its derivatives, vinyl ethers and vinylpyrrolidone.

the salts of polystyrenesulfonic acid, the sodium salts having a molecular weight of about 500,000 and about 100,000 sold respectively under the names Flexan 500 and Flexan 130 by National Starch. These compounds are described in patent FR 2,198,719.

the salts of polyacrylamidesulfonic acids, those mentioned in U.S. Pat. No. 4,128,631 and more particularly polyacrylamidoethylpropanesulfonic acid sold under the name COSMEDIA POLYMER HSP 1180 by Henkel.

According to the invention, the anionic fixing polymers are preferably selected from acrylic acid copolymers such as-the acrylic acid/ethyl acrylate/N-tert-butylacrylamide terpolymer sold under the name ULTRAHOLD STRONG by the company BASF, the copolymers derived from crotonic acid such as the vinyl acetate/vinyl tert-butyl benzoate/crotonic acid terpolymers and the crotonic acid/vinyl acetate/vinyl neododecanoate terpolymers sold under the name Resine 28-29-30 by the company NATIONAL STARCH, the polymers derived from maleic, fumaric, itaconic acids or anhydrides with vinyl esters, vinyl ethers, vinyl halides, phenylvinyl derivatives, acrylic acid and its esters such as the monoesterified methyl vinyl ether/maleic anhydride copolymer sold under the name GANTREZ ES 425 by the company ISP, the copolymers of methacrylic acid and methyl methacrylate sold under the name EUDRAGIT L by the company ROHM PHARMA, the copolymer of methacrylic acid and ethyl acrylate sold under the name LUVIMER MAEX or MAE by the company BASF, the vinyl acetate/crotonic acid copolymer sold under the name LUVISET CA 66 by the company BASF and the vinyl acetate/crotonic acid copolymer grafted by polyethyleneglycol under the name ARISTOFLEX A by the company BASF.

The most particularly preferred anionic fixing polymers are selected from the monoesterified methyl vinyl ether/maleic anhydride copolymer sold under the name GANTREZ ES 425 by the company ISP, the acrylic acid/ethyl acrylate/N-tert-butylacrylamide terpolymer sold under the name ULTRAHOLD STRONG by the company BASF, the methacrylic acid and methyl methacrylate copolymers sold under the name EUDRAGIT L by the company ROHM PHARMA, the vinyl acetate/vinyl tert-butyl benzoate/crotonic acid terpolymers and crotonic acid/vinyl acetate/vinyl neododecanoate terpolymers sold under the name Résine 28-29-30 by the company NATIONAL STARCH, the methacrylic acid and ethyl acrylate copolymer sold under the name LUVIMER MAEX OR MAE by the company BASF, the vinylpyrrolidone/acrylic acid/lauryl methacrylate terpolymer sold under the name ACRYLIDONE LM by the company ISP.

The amphoteric fixing polymers which can be used in accordance with the invention can be selected from the polymers comprising units B and C distributed randomly in the polymer chain where B denotes a unit deriving from a monomer comprising at least one basic nitrogen atom and C denotes a unit deriving from an acid monomer comprising one or several carboxylic or sulfonic groups or B and C can denote groups deriving from zwitterionic monomers of carboxybetaines or sulfobetaines;

B and C can also denote a cationic polymer chain comprising primary, secondary, tertiary or quaternary amine groups, in which at least one of the amine groups has a carboxylic or sulfonic group linked by means of a hydrocarbon radical or B and C form part of a chain of a polymer with an α,β-dicarboxylic ethylene unit, one of the carboxylic groups of which has been caused to react with a polyamine comprising one or several primary or secondary amine groups.

The more particularly preferred amphoteric fixing polymers corresponding to the definition given above are selected from the following polymers:

(1) the polymers resulting from the copolymerization of a monomer derived from a vinyl compound having a carboxylic group such as more particularly acrylic acid, methacrylic acid, maleic acid, alpha-chloroacrylic acid, and a basic monomer derived from a substituted vinyl compound containing at least one basic atom such as more particularly dialkylaminoalkylmethacrylate and acrylate, dialkylaminoalkylmethacrylamide and acrylamide. Such compounds are described in American U.S. Pat. No. 3,836,537.

(2) the polymers comprising units deriving:

a) from at least one monomer selected from the acrylamides or methacrylamides substituted on the nitrogen by an alkyl radical,

b) from at least one acid comonomer containing one or several reactive carboxylic groups, and

c) from at least one basic comonomer such as esters with primary, secondary, tertiary and quaternary amine substituents of acrylic and methacrylic acids and the quaternization product of dimethylaminoethyl methacrylate with dimethyl or diethyl sulfate.

The more particularly preferred N-substituted acrylamides or methacrylamides according to the invention are the groups the alkyl radicals of which contain 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acid comonomers are selected more particularly from acrylic, methacrylic, crotonic, itaconic, maleic, fumaric acids and the alkyl monoesters having 1 to 4 carbon atoms of maleic or fumaric acids or anhydrides.

The preferred basic comonomers are aminoethyl, butyl aminoethyl, N,N′-dimethylaminoethyl, N-tert-butylaminoethyl methacrylates.

Copolymers the CTFA name (4^{th} Ed., 1991) of which is octylacrylamide/acrylates/butylaminoethyl methacrylate copolymer such as the products sold under the name AMPHOMER or LOVOCRYL 47 by the company NATIONAL STARCH are particularly used.

(3) crosslinked and alkylated polyamino amides deriving partially or totally from polyaminoamides of general formula III:

[CO—R₁₀—CO—Z] (III)

in which R₁₀ represents a divalent radical derived

from a saturated dicarboxylic acid, an ethylene double bond mono- or dicarboxylic aliphatic acid, an ester of a lower alkanol having 1 to 6 carbon atoms of these acids or a radical deriving from the addition of any one of said acids with a bis primary or bis secondary amine, and Z denotes a radical of a bis-primary, mono- or bis-secondary polyalkylene-polyamine and preferably represents:

a) in proportions of 60 to 100 mol %, the radical IV

—NH[(CH₂)ₓ—NH]ₚ (IV)

where x=2 and p=2 or 3, or x=3 and p=2

this radical deriving from diethylene triamine, triethylene tetraamine or dipropylene triamine;

b) in proportions of 0 to 40 mol %, the radical (IV) above, in which x=2 and p=1 and which derives from ethylenediamine, or the radical deriving from piperazine

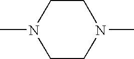

c) in proportions of 0 to 20 mol %, the radical —NH—(CH₂)₆—NH— deriving from hexamethylenediamine, these polyaminoamines being crosslinked by addition of a bifunctional crosslinking agent selected from epihalohydrines, diepoxides, dianhydrides, bis unsaturated derivatives, by means of 0.025 to 0.35 mol of crosslinking agent per amine group of the polyaminoamide and alkylated by action of acrylic acid, chloroacetic acid or an alkane sultone or their salts.

The saturated carboxylic acids are selected preferably from acids having 6 to 10 carbon atoms such as adipic, trimethyl-2,2,4-adipic and trimethyl-2,4,4-adipic, terephthalic acid, acids with a double ethylene bond such as for example acrylic, methacrylic, itaconic acids.

The alkane sultones used in alkylation are preferably propane or butane sultone, the salts of alkylation agents are preferably the sodium or potassium salts.

(4) the polymers comprising zwitterionic units of formula V:

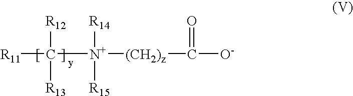

in which R₁₁ denotes a polymerizable unsaturated group such as an acrylate, methacrylate, acrylamide or methacrylamide group, y and z represents a whole number from 1 to 3, R₁₂ and R₁₃ represent a hydrogen, methyl, ethyl or propyl atom, R₁₄ and R₁₅ represent a hydrogen atom or an alkyl radical such that the sum of the carbon atoms in R₁₄ and R₁₅ does not exceed 10.

The polymers comprising such units can also comprise units derived from non-zwitterionic monomers such as the dimethyl or diethylaminoethyl acrylate or methacrylate or alkyl acrylates or methacrylates, acrylamides or methacrylamides or vinyl acetate.

By way of example can be cited the butyl methacrylate/dimethyl carboxymethylammonio ethyl methacrylate copolymer such as the product sold under the name DIAFORMER Z301 by the company SANDOZ.

(5) the polymers derived from chitosan comprising monomer units corresponding to the following formulae:

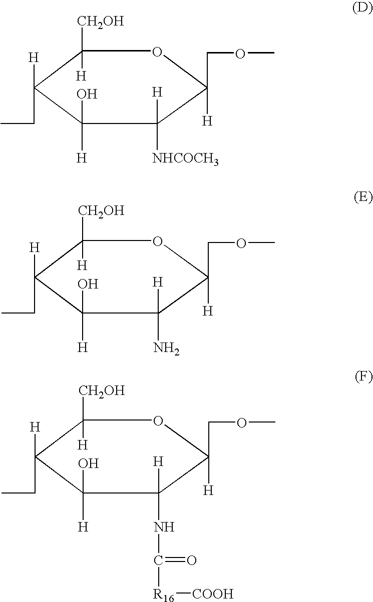

the unit D being present in proportions of between 0 and 30%, unit E in proportions of between 5 and 50% and unit F in proportions of between 30 and 90%, it being understood that in this unit F, R₁₆ represents a radical of formula:

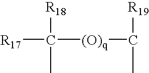

in which if q=0, R₁₇, R₁₈ and R₁₉, identical or different, each represent a hydrogen atom, a methyl, hydroxyl, acetoxy or amino residue, a monoalkylamine residue or a dialkylamine residue possibly interrupted by one or several nitrogen atoms and/or possibly substituted by one or several amine, hydroxyl, carboxyl, alkylthio, sulfonic groups, an alkylthio residue the alkyl group of which has an amino residue, one at least of the radicals R₁₇, R₁₈ and R₁₉ being in this case a hydrogen atom;

or if q=1, R₁₇, R₁₈ and R₁₉ each represent a hydrogen atom, and the salts formed by these compounds with bases or acids.

(6) The polymers derived from the N-carboxyalkylation of chitosan such as N-carboxymethyl chitosan or N-carboxybutyl chitosan sold under the name “EVALSAN” by the company JAN DEKKER.

(7) The polymers corresponding to the general formula (VI) for example described in French patent 1 400 366:

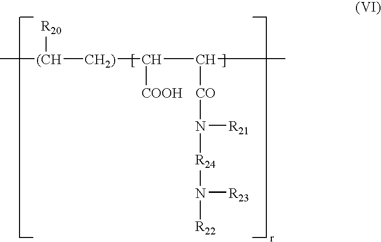

in which R₂₀ represents a hydrogen atom, a CH₃O, CH₃CH₂O phenyl radical, R₂₁ denotes hydrogen or a lower alkyl radical such as methyl, ethyl, R₂₂ denotes hydrogen or a lower alkyl radical such as methyl, ethyl, R₂₃ denotes a lower alkyl radical such as methyl, ethyl or a radical corresponding to the formula: —R₂₄—N(R₂₂)₂, R₂₄ representing a —CH₂CH₂—, —CH₂—CH₂—CH₂—, —CH₂—CH(CH₃)— group, R₂₂ having the meanings mentioned above,

and the higher homologs of these radicals and containing up to 6 carbon atoms.

(8) Amphoteric polymers of the type -D-X-D-X- selected from:

a) the polymers obtained by action of chloroacetic acid or sodium chloroacetate on the compounds comprising at least one unit of formula:

-D-X-D-X-D- (VII)

where D denotes a radical

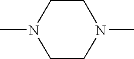

and X denotes the symbol E or E′, E or E′ identical or different denote a divalent radical which is a straight-chain or branched alkylene radical comprising up to 7 carbon atoms in the principal chain non-substituted or substituted by hydroxyl groups and being able to comprise moreover oxygen, nitrogen, sulfur atoms, 1 to 3 aromatic and/or heterocyclic cycles; oxygen, nitrogen and sulfur atoms being present in the form of ether, thioether, sulfoxide, sulfone, sulfonium, alkylamine, alkenylamine groups, hydroxyl, benzylamine, amine oxide, quaternary ammonium, amide, mide, alcohol, ester and/or urethane groups.

b) The polymers of formula:

-D-X-D-X- (VII′)

where D denotes a radical

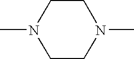

and X denotes the symbol E or E′ and at least once E′; E having the meaning indicated above and E′ is a divalent radical which is a straight-chain or branched alkylene radical having up to 7 carbon atoms in the principal chain, substituted or not by one or several hydroxyl radicals and comprising one or several nitrogen atoms, the nitrogen atom being substituted by an interrupted alkyl chain possibly by an oxygen atom and comprising necessarily one or several carboxyl functions or one or several hydroxyl and betainized functions by reaction with chloroacetic acid or sodium chloroacetate.

(9) the copolymers alkyl(C1-C5) vinyl ether/maleic anhydride modified partially by semi-amidification with an N,N-dialkylaminoalkylamine such as N,N-dimethylaminopropylamine or by semi-esterification with an N,N-dialkanolamine. These copolymers can also comprise other vinyl comonomers such as vinylcaprolactam.

The amphoteric fixing polymers particularly preferred according to the invention are those of family (3) such as the copolymers, the CTFA name of which is Octylacrylamide/acrylates/butylaminoethyl methacrylate copolymer such as the products sold under the names AMPHOMER, AMHOMER LV 71 or LOVOCRYL 47 by the company NATIONAL STARCH and those. of family (4) such as the butyl methacrylate/dimethyl-carboxymethylammonioethyl methacrylate copolymer for example sold under the name DIAFORMER Z301 by the company SANDOZ.

The nonionic fixing polymers which can be used according to the present invention are selected for example from:

the homopolymers or copolymers of vinylpyrrolidone or vinylcaprolactam;

the copolymers of vinylpyrrolidone and vinyl acetate;

polyalkyloxazolines such as the polyethyloxazolines proposed by the company DOW CHEMICAL under the names PEOX 50 000, PEOX 200 000 and PEOX 500 000;

the homopolymers of vinyl acetate such as the product proposed under the name APPRETAN EM by the company HOECHST or the product proposed under the name RHODOPAS A 012 by the company RHONE POULENC;

the copolymers of vinyl acetate and acrylic ester such as the product proposed under the name RHODOPAS AD 310 of RHONE POULENC;

the copolymers of vinyl acetate and ethylene such as the product proposed under the name APPRETAN TV by the company HOECHST;

the copolymers of vinyl acetate and maleic ester for example dibutyl maleate such as the product proposed under the name APPRETAN MB EXTRA by the company HOECHST;

the copolymers of polyethylene and maleic anhydride;

the homopolymers of alkyl acrylates and the homopolymers of alkyl methacrylates such as the product proposed under the name MICROPEARL RQ 750 by the company MATSUMOTO or the product proposed under the name LUHYDRAN A 848 S by the company BASF;

the copolymers of acrylic esters such as for example the copolymers of alkyl acrylates and alkyl methacrylates such as the products proposed by the company ROHM&HAAS under the names PRIMAL AC-261 K and EUDRAGIT NE 30 D, by the company BASF under the names ACRONAL 601, LUHYDRAN LR 8833 or 8845, by the company HOECHST under the names APPRETAN N 9213 or N9212;

the copolymers of acrylonitrile and a nonionic monomer selected for example from butadiene and the alkyl (meth)acrylates; the products proposed under the names NIPOL LX 531 B by the company NIPPON ZEON or those proposed under the name CJ 0601 B by the company OHM & HAAS can be cited;

polyurethanes such as the products proposed under the names ACRYSOL RM 1020 or ACRYSOL RM 2020 by the company ROHM & HAAS, the products URAFLEX XP 401 UZ, URAFLEX XP 402 UZ by the company DSM RESINS;

the copolymers of alkyl acrylate and urethane such as the product 8538-33 by the company NATIONAL STARCH;

polyamides such as the product ESTAPOR LO 11 proposed by the company RHONE POULENC.

The alkyl radicals of the nonionic polymers have 1 to 6 carbon atoms unless stated to the contrary.

The nonionic polymers which are very particularly suitable for achieving the compositions in accordance with the invention are those selected from:

the copolymers of vinyllactam such as the copolymers of vinylpyrrolidone and vinyl acetate and the vinylpyrrolidone/vinyl acetate/vinyl propionate copolymers

polyvinylcaprolactam LUVISKOL PLUS (BASF)

the homopolymers of vinyl acetate such as APPRETAN EM (HOECHST) or RHODOPAS A 012 (RHONE POULENC)

polyalkyloxazolines such as PEOX 50 000 and PEOX 500 000 (DOW CHEMICAL)

the copolymers of vinyl acetate and acrylic ester such as RHODOPAS AD 310 (RHONE POULENC)

the copolymers of vinyl acetate and ethylene such as APPRETAN TV (HOECHST)

the copolymers of vinyl acetate and maleic ester such as APPRETAN MB EXTRA (HOECHST)

the homopolymers of alkyl acrylates and the homopolymers of alkyl methacrylates such as LUHYDRAN A 848 S (BASF)

the copolymers of acrylic esters such as PRIMAL AC-261 K (ROHM & HAAS), ACRONAL 601 (BASF) or APPRETAN N 9213 (HOECHST)

the copolymers of acrylonitrile and a nonionic monomer such as CJ 0601 B (ROHM & HAAS)

polyurethanes such as ACRYSOL RM 1020 or ACRYSOL RM 2020 (ROHM & HAAS)

the copolymers of alkyl acrylate and urethane such as 8538-33 (NATIONAL STARCH)

polyamides such as ESTAPOR LO 11 (RHONE POULENC)

According to the invention, fixing polymers of the grafted siliconized type comprising a polysiloxane portion and a portion constituted of a non-siliconized organic chain, one of the two portions constituting the principal chain of the polymer, the other being grafted on said principal chain, can also be used. These polymers are for example described in patent applications EP-A-0 412 704, EP-A-0 412 707, EP-A-0 640 105 and WO 95/00578, EP-A-0582 152 and WO 93/23009 and U.S. Pat. No. 4,693,935, U.S. Pat. No. 4,728,571 and U.S. Pat. No. 4,972,037. These polymers are preferably anionic or nonionic.

Such polymers are for example copolymers likely to be obtained by radical polymerization from the mixture of monomers constituted by:

a) 50 to 90% by weight tert-butyl acrylate;

b) 0 to 40% by weight acrylic acid;

c) 5 to 40% by weight siliconized macromer of formula:

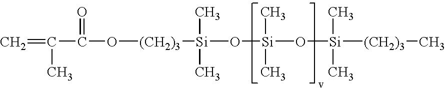

with v being a number ranging from 5 to 700; the percentages by weight being calculated in relation to the total weight of the monomers.

Other examples of grafted siliconized polymers are notably polydimethylsiloxanes (PDMS) on which are grafted, by means of a connecting link of the thiopropylene type, mixed polymer units of the poly(meth)acrylic acid type and of the alkyl poly(meth)acrylate type and polydimethylsiloxanes (PDMS) on which are grafted, by means of a connecting link of the thiopropylene type, polymer units of the isobutyl poly(meth)acrylate type.

Polyurethanes functionalized or not, siliconized or not, can also be used as fixing polymers. As a polyurethane which can be used within the context of the invention, the product marketed under the name LUVISET PUR by BASF can be cited.

The polyurethanes particularly aimed at by the present invention are those described in patents EP 0 751 162, EP 0 637 600, FR 2 743 297 and EP 0 648 485, of which the Applicant is Proprietor, and patents EP 0 656 021 or WO 94/03510 of the Company BASF and EP 0 619 111 of the Company National Starch.

Preferably, anionic or nonionic polymers are selected as fixing polymers.

Of the cationic polymers can be cited more particularly polymers of the polyamine, polyaminoamide and quaternary polyammonium type. These are known products.

Polymers of the polyamine, polyaminoamide, quaternary polyammonium type, which can be used in accordance with the present invention, which can be notably mentioned, are those described in French patents No. 2 505 348 or 2 542 997. Of these polymers can be cited:

(1) the homopolymers or copolymers derived from acrylic or methacrylic amides or esters and comprising at least one of the units of following formulae:

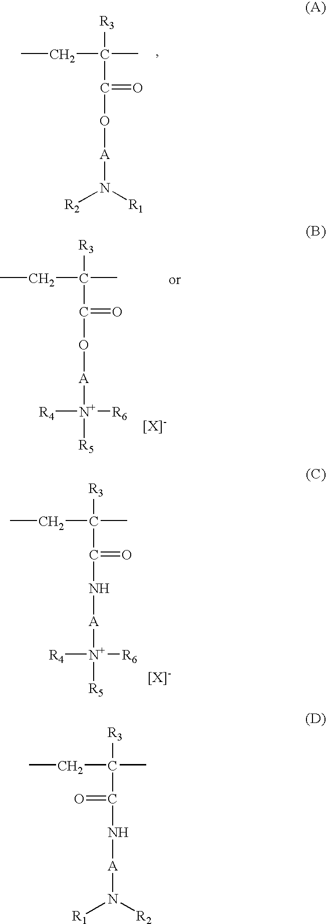

in which:

R₃, identical or different, denote a hydrogen atom or a CH₃ radical;

A, identical or different, represent an alkyl group, linear or branched, with 1 to 6 carbon atoms, preferably 2 or 3 carbon atoms or a hydroxylalkyl group with 1 to 4 carbon atoms;

R₄, R₅, R₆, identical or different, represent an alkyl group having 1 to 18 carbon atoms or a benzyl radical and preferably an alkyl group having 1 to 6 carbon atoms;

R₁ and R₂, identical or different, represent hydrogen or an alkyl group having 1 to 6 carbon atoms and preferably methyl or ethyl;

X denotes an anion derived from a mineral or organic acid such as a methosulfate anion or a halide such as chloride or bromide.

The copolymers of family (1) can contain furthermore one or several units deriving from comonomers which can be selected from the family of acrylamides, methacrylamides, diacetone acrylamides, acrylamides and methacrylamides substituted on the nitrogen by lower alkyls (C1-C4), acrylic or methacrylic acids or their esters, vinyllactams such as vinylpyrrolidone or vinylcaprolactam, vinyl esters.

Thus, of these copolymers of family (1), can be cited:

copolymers of acrylamide and dimethylaminoethyl methacrylate quaternized with dimethyl sulfate or with a dimethyl holide such as that sold under the name HERCOFLOC by the company HERCULES,

copolymers of acrylamide and methyacryloyloxyethyltrimethylammonium chloride described for example in patent application EP-A-080976 and sold under the name BINA QUAT P 100 by the company CIBA GEIGY,

the copolymer of acrylamide and methyacryloyloxyethyltrimethylammonium methosulfate sold under the name RETEN by the company HERCULES,

the vinylpyrrolidone/dialkylaminoalkyl acrylate or methacrylate copolymers quaternized or not, such as the products sold under the name “GAFQUAT” by the company ISP such as for example “GAFQUAT 734” or “GAFQUAT 75511” or the products named “COPOLYMER 845, 958 and 937”. These polymers are described in detail in French patents 2,077,143 and 2,393,573,

dimethyl amino ethyl methacrylate/vinylcaprolactam/vinylpyrrolidone terpolymers such as the product sold under the name GAFFIX VC 713 by the company ISP,

the vinylpyrrolidone/methacrylamidopropyl dimethylamine copolymer marketed notably under the name STYLEZE CC 10 by ISP.

and the quaternized dimethylaminopropyl methacrylamide/vinylpyrrolidone copolymers such as the product sold under the name “GAFQUAT HS 100” by the company ISP.

(2) The derivatives of cellulose ethers comprising quaternary ammonium groups described in French patent 1 492 597, and in particular the polymers marketed under the names “JR” (JR 400, JR 125, JR 30M) or “LR” (LR 400, LR 30M) by the Company Union Carbide Corporation. These polymers are also defined in the CTFA dictionary as quaternary hydroxyethylcellulose ammoniums having reacted with an epoxide substituted by a trimethylammonium group.

(3) The cationic cellulose derivatives such as the cellulose copolymers or the cellulose derivatives grafted with a water-soluble quaternary ammonium monomer, and described notably in U.S. Pat. No. 4,131,576, such as hydroxyalkyl celluloses, such as the hydroxymethyl-, hydroxyethyl- or hydroxypropyl- celluloses grafted notably with a methacryloylethyl trimethylammonium, methacrylmidopropyl trimethylammonium, dimethyldiallylammonium salt.

The marketed products corresponding to this definition are more particularly the products sold under the name “Celquat L 200” and “Celquat H 100” by the Company National Starch.

(4) The cationic polysaccharides described more particularly in U.S. Pat. Nos. 3,589,578 and 4,031,307 such as guar gums containing cationic trialkylammonium groups. Guar gums modified by a salt (for example chloride) of 2,3-epoxypropyl trimethylammonium are used for example.

Such products are marketed notably under the tradenames JAGUAR C13 S, JAGUAR C 15, JAGUAR C 17 or JAGUAR C162 by the company MEYHALL.

(5) polymers constituted of piperazinyl units and of divalent alkylene or hydroxyalkylene radicals, straight-chain or branched, possibly interrupted by atoms of oxygen, sulfur, nitrogen or by aromatic or heterocyclic rings, and the oxidation and/or quaternization products of these polymers. Such polymers are notably described in French patents 2,162,025 and 2,280,361;

(6) polyaminoamides soluble in water prepared in particular by polycondensation of an acid compound with a polyamine; these polyaminoamides can be crosslinked by an epihalohydrin, a diepoxide, a dianhydride, a non-saturated dianhydride, a bis-unsaturated derivative, a bis-halohydrin, a bis-azetidinium, a bis-haloacyldiamine, an alkyl bis-halide or even by an oligomer resulting from the reaction of a reactive bifunctional compound in relation to a bis-halohydrin, a bis-azetidinium, a bis-haloacyldiamine, an alkyl bis-halide, an epihalohydrin, a diepoxide or a bis-unsaturated derivative; the crosslinking agent being used in proportions ranging from 0.025 to 0.35 mol per amine group of the polymaoamide; these polyaminoamides can be alkylated or if they comprise one or several tertiary amine functions, quaternized. Such polymers are notably described in French patents 2 252,840 and 2,368,508;

(7) the derivatives of polyaminoamides resulting from the condensation of polyalkylene polyamines with polycarboxylic acids followed by alkylation by bifunctional agents. The adipic acid-diakylaminohydroxyalkyldialylene triamine polymers in which the alkyl radical comprises 1 to 4 carbon atoms and denotes preferably methyl, ethyl, propyl, can be cited for example. Such polymers are notably described in French patent 1,583,363.

Of these derivatives can be cited more particularly the adipic acid/dimethylaminohydroxypropyl/diethylene triamine polymers sold under the name “Cartaretine F, F4 or F8” by the company Sandoz.

(8) the polymers obtained by reaction of a polyalkylene polyamine comprising two primary amine groups and at least one secondary amine group with a dicarboxylic acid selected from diglycolic acid and saturated aliphatic dicarboxylic acids having 3 to 8 carbon atoms. The molar ratio between the polyalkylene polylamine and the dicarboxylic acid being between 0.8:1 and 1.4:1; the resulting polyaminoamide being caused to react with epichlorohydrin in a molar ratio of epichlorohydrin in relation to the secondary amine group of the polyaminoamide of between 0.5:1 and 1.8:1. Such polymers are notably described in American U.S. Pat. Nos. 3,227,615 and 2,961,347.

Polymers of this type are in particular marketed under the name “Hercosett 57” by the company Hercules Inc. or under the name “PD 170” or “Delsette 101” by the company Hercules in the case of the adipic acid/epoxypropyl/diethylene-triamine copolymer.

(9) the cyclopolymers of alkyl diallyl amine or dialkyl diallyl ammonium such as the homopolymers or copolymers comprising as principal constituent of the chain units corresponding to formulae (VI) or (VI′):

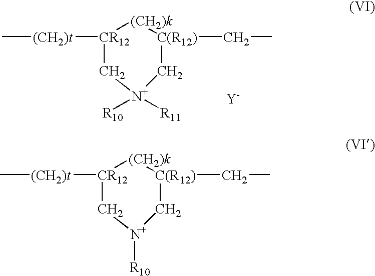

formulae in which k and t are equal to 0 or 1, the sum of k+t being equal to 1, R₁₂ denotes a hydrogen atom or a methyl radical; R₁₀ and R₁₁, independently of one another, denote an alkyl group having 1 to 22 carbon atoms, a hydroxyalkyl group in which the alkyl group has preferably 1 to 5 carbon atoms, a lower amidoalkyl group (C1-C4) or R₁₀ and R₁₁ can denote jointly with the nitrogen atom to which they are attached, heterocyclic groups such as piperidinyl or morpholinyl; Y⁻ is an anion such as bromide, chloride, acetate, borate, citrate, tartrate, bisulfate, bisulfite, sulfate, phosphate. These polymers are notably described in French patent 2,080,759 and in its certificate of addition 2,190,406.

R₁₀ and R₁₁, independently of one another, denote preferably an alkyl group having 1 to 4 carbon atoms.

Of the polymers defined above can be cited more particularly the homopolymer of dimethyldiallylammonium chloride sold under the name “Merquat 100” by the company Calgon (and its homologs with low weight-average molecular weights) and the copolymers of diallyldimethylammonium chloride and acrylamide marketed under the name “MERQUAT 550”.

(10) the quaternary diammonium polymer containing recurrent units corresponding to the formula:

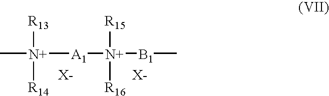

formula (VII) in which:

R13, R14, R15 and R16, identical or different, represent aliphatic, alicyclic or arylaliphatic radicals containing 1 to 20 carbon atoms or lower hydroxyalkylaliphatic radicals, or R13, R14, R15 and R16, together or separately, constitute with the nitrogen atoms to which they are attached heterocycles containing possibly a second heteroatom other than nitrogen or R13, R14, R15 and R16 represent a linear or branched C1-C6 alkyl radical substituted by a nitrile, ester, acyl, amide or —CO—O—R17-D or —CO—NH—R17-D group where R17 is an alkylene and D a quaternary ammonium group;

A1 and B1 represent polymethylene groups containing 2 to 20 carbon atoms which can be linear or branched, saturated or unsaturated, and can contain, linked to or interposed in the principal chain, one or several aromatic cycles, or one or several oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A1, R13 and R15 can form with the two nitrogen atoms to which they are attached a piperazine cycle;

moreover if A1 denotes a linear or branched, saturated or unsaturated alkylene or hydroxyalkylene radical, B1 can also denote a (CH2)n-CO-D-OC—(CH2)n- group in which D denotes:

a) a glycol residue of formula: —O-Z-O—, where Z denotes a linear or branched hydrocarbon radical or a group corresponding to one of the following formulae;

—(CH2-CH2-O)x-CH2-CH2-

—[CH2-CH(CH3)-O]y-CH2-CH(CH3)-

where x and y denote a whole number from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon radical, or the divalent radical

—CH2-CH2-S—S-CH2-CH2-;

d) a ureylene group of formula: —NH—CO—NH—;

Preferably, X⁻ is an anion such as chloride or bromide.

These polymers have a number-average molecular weight generally of between 1000 and 100,000.

Polymers of this type are notably described in French patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

More particularly the polymers can be used which are constituted of recurrent units corresponding to the formula:

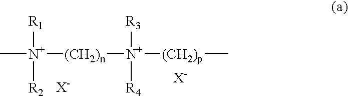

in which R₁, R₂, R₃ and R₄, identical or different, denote an alkyl or hydroxyalkyl radical having about 1 to 4 carbon atoms, n and p are whole numbers varying from about 2 to 20 and, X⁻ is an anion derived from a mineral or organic acid.

A particularly preferred compound of formula (a) is that for which R₁, R₂, R₃ and R₄ represent a methyl radical and n=3, p=6 and X=Cl, named Hexadimethrine chloride according to the INCI (CTFA) nomenclature.

(11) quaternary polyammonium polymers constituted of units of formula (VIII):

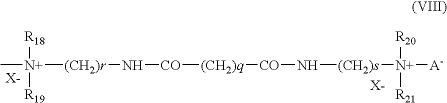

formula in which:

R18, R19, R20 and R21, identical or different, represent a hydrogen atom or a methyl, ethyl, propyl, β-hydroxyethyl, β-hydroxypropyl or —CH2CH2(OCH2CH2)pOH radical,

where p is equal to 0 or to a whole number between 1 and 6, provided that R18, R19, R20 and R21 do not represent simultaneously a hydrogen atom,

r and s, identical or different, are whole numbers between 1 and 6,

q is equal to 0 or to a whole number between 1 and 34,

X denotes a halogen atom,

A denotes a radical of a dihalide or represents preferably —CH2-CH2-O-CH2-CH2-.

Such compounds are notably described in patent application EP-A-122 324.

Of these can be cited for example the products “Mirapol A 15”, “Mirapol AD1”, “Mirapol AZ1” and “Mirapol 175” sold by the company Miranol.

(12) The quaternary polymers of vinylpyrrolidone and vinylimidazole such as for example the products marketed under the names Luviquat FC 905, FC 550 and FC 370 by the company B.A.S.F.

(13) Polyamines such as Polyquart H sold by HENKEL, referred to under the name “POLYETHYLENEGLYCOL (15) TALLOW POLYAMINE” in the CTFA dictionary.

(14) Crosslinked polymers of salts of methacryloyloxyalkyl (C₁-C₄) trialkyl (C₁-C₄) ammonium such as the polymers obtained by homopolymerization of the dimethylaminoethylmethacrylate quaternized by methyl chloride, or by copolymerization of acrylamide with dimethylaminoethylmethacrylate quaternized by methyl chloride, homo- or copolymerization being followed by crosslinking by an olefinic nonsaturation compound, in particular methylenebisacrylamide. More particularly a crosslinked acrylamide/methacryloyloxyethyl trimethylammonium chloride copolymer (20/80 by weight) can be used in the form of a dispersion containing 50% by weight of said copolymer in mineral oil. This dispersion is marketed under the name “SALCARE® SC 92” by the Company ALLIED COLLOIDS. A crosslinked homopolymer of methacryloyloxyethyl trimethylammonium chloride containing about 50% by weight of the homopolymer in mineral oil or in a liquid ester can also be used. These dispersions are marketed under the names “SALCARE® SC 95” and “SALCARE® SC 96” by the Company ALLIED COLLOIDS.

Other cationic polymers which can be used within the context of the invention are polyalkyleneimines, in particular polyethyleneimines, polymers containing vinylpyridine or vinylpyridinium units, condensates of polyamines and epichlorohydrin, quaternary polyureylenes and derivatives of chitin.

Of the cationic polymers likely to be used within the context of the present invention, it is preferred to use the quaternary derivatives of cellulose ether such as the products sold under the name “JR 400” by the company UNION CARBIDE CORPORATION, the cyclopolymers, in particular the polymers or copolymers of dimethyldiallylammonium chloride and acrylamide, sold under the names “MERQUAT 100”, “MERQUAT 550” and “MERQUAT S” by the Company CALGON, cationic polysaccharides such as guar gums modified by 2,3-epoxypropyl trimethylammonium chloride marketed for example under the name “JAGUAR C13S” by the company MEYHALL and the vinylpyrrolidone/methacrylamidopropyl dimethylamine copolymer marketed notably under the name STYLEZE CC 10 by the company ISP and their mixtures. Polyurethanes, functionalized or not, siliconized or not, can also be used as fixing polymers. As polyurethane which can be used within the context of the invention, the products marketed under the name LUVISET PUR and LUVISET Si PUR by BASF can be cited.

Thickener

Within the meaning of the present invention, by “thickener” is understood any compound which, at 1% by weight in water at a temperature of 25° C., leads to a viscosity measured by means of a Rheomat RM180 greater than 0.1 Pa.s, and more advantageously still at 0.2 Pa.s, at a shear rate of 200 s⁻¹.

The invention encompasses natural and synthetic thickeners. As natural thickener can be cited celluloses and their derivatives, xanthan, scleroglucan, gellan, rhamsan gums, alginates, maltodextrin, starch and its derivatives, karaya gum, carob flour and guar gums.

Synthetic thickening agents according to the invention are advantageously polymers or copolymers of acrylic and/or methacrylic acid, such as acrylic acid/ethyl acrylate copolymers and carboxyvinyl polymers. Examples of such polymers or copolymers are notably the “carbomers” (CTFA) sold by the company GOODRICH under the name Carbopol or the glyceryl polymethacrylate marketed by the company GUARDIAN under the name Lubragel or even the glyceryl polyacrylate marketed under the name Hispagel by the company HISPANO CHIMICA.

As other acrylic compounds the copolymers of acrylic or methacrylic acid comprising at least one C1 to C30 alkyl acrylate unit and/or one urethane unit possibly substituted by a fatty chain can be cited. In particular Pemulen TRl (Goodrich), Viscophobe DB 1000 (Union Carbide) can be cited.

Polyethyleneglycols (PEG) and their derivatives can even be used as thickening agent.

Thickening polyacrylamides can also be used advantageously as thickening agent. These can more particularly be selected from:

crosslinked homopolymers of 2-acrylamido-2-methylpropanesulfonic,

possibly crosslinked copolymers of acrylamide and ammonium acrylate,

possibly crosslinked copolymers of acrylamide (or methacrylamide) and methacryloyloxyethyl trimethylammonium chloride,

possibly crosslinked partially or totally neutralized copolymers. of acrylamide and 2-acrylamido 2-methyl propanesulfonic acid.

As crosslinked acrylamide/ammonium acrylate copolymers, used in accordance with the present invention, acrylamide/ammonium acrylate copolymers (5/95 by weight) crosslinked by a crosslinking agent with olefinic polyunsaturation, such as divinylbenzene, tetraallyloxyethane, methylene bis-acrylamide, diallyl ether, polyallylpolyglyceryl ethers or allyl ethers of alcohol of the series of sugars, such as erythritol, pentaerythritol, arabitol, mannitol, sorbitol or glucose can be more particularly cited.

Similar copolymers are described and prepared in French patent FR-2,416,723 and U.S. Pat. No. 2,798,053 and U.S. Pat. No. 2,923,692.

This type of crosslinked copolymer is used in particular in the form of a water-in-oil emulsion constituted of about 30% by weight of said copolymer, 25% by weight of paraffin oil, 4% by weight of a mixture of sorbitan stearate and a hydrophilic ethoxylated derivative and 41% by weight of water. Such an emulsion is marketed under the name “BOZEPOL C” by the Company HOECHST.

The copolymers of acrylamide and sulfonic 2-acrylamido 2-methyl propane acid, used in accordance with the present invention, are copolymers crosslinked by an olefinic polyunsaturation compound, such as those mentioned previously, and partially or totally neutralized by a neutralization agent such as soda, potassium, ammonia or an amine such as triethanolamine or monoethanolamine.

They can be prepared by copolymerizing acrylamide and sodium 2-acrylamido 2-methylpropane sulfonate by radical route by means of initiators of the azobisisobutyronitrile type and by precipitation in an alcohol such as tert-butanol.

Copolymers obtained by copolymerization of 70 to 55 mol % acrylamide and 30 to 45 mol % sodium 2-acrylamido 2-methylpropane sulfonate are used more particularly. The crosslinking agent being used at concentrations of 10⁻⁴ to 4.10⁻⁴ mol per mole of the monomer mixture.

These particular copolymers are incorporated in the compositions of the invention, preferentially, in the form of water-in-oil emulsions containing 35 to 40% by weight of this copolymer, 15 to 25% by weight of a mixture of C₁₂-C₁₃ isoparaffinic hydrocarbons, 3 to 8% by weight polyethyleneglycol lauryl ether with 7 mol ethylene oxide and water. Such an emulsion is marketed under the name “SEPIGEL 305” by the company SEPPIC.

The crosslinked copolymer of acrylamide and methacryloyl oxyethyl trimethylammonium chloride, used according to the invention, is more particularly a copolymer obtained by copolymerization of acrylamide and dimethylaminoethylmethacrylate quaternized by methyl chloride, followed by crosslinking by an olefinic nonsaturation compound, in particular methylene-bis-acrylamide.

A crosslinked acrylamide/methacryloyl oxyethyl trimethylammonium chloride copolymer (around 50/50 by weight) in the form of a dispersion containing 50% by weight of said copolymer in mineral oil is used more particularly. This dispersion is marketed under the name “SALCARE SC92” by the Company ALLIED COLLOIDS.

The non-crosslinked copolymers of methacrylamide and methacryloyloxyethyl trimethylammonium chloride are for example the products sold under the tradenames ROHAGIT KF 400 and KF720 by the company ROHM and Haas.

As other synthetic derivatives can be cited the fatty chain thickening polymers derived from cellulose, nonionic (NATROSOL PLUS from HERCULE) or cationic (QUADRISOFT UM 200) or nonionic polyurethanes (ACULYN 44 or 46).

In accordance with the invention, the polymer(s) (ii) selected from the fixing, thickening polymers or a mixture thereof is advantageously present in the cosmetic composition in a quantity of between 0.01 and 20% and more preferentially of between 0.1 and 8% in relation to the total weight of the composition.

Composition

The cosmetically acceptable medium comprises water and/or a cosmetically acceptable solvent notably selected from the lower C₁-C₄ alcohols, such as ethanol, isopropanol, tert-butanol and n-butanol; polyols, such as propyleneglycol; polyol ethers; acetone and their mixtures.

The preferred cosmetically acceptable medium according to the invention is a hydroalcohol medium, containing advantageously water and etahnol.

The quantity of water is, preferably, between 10 and 95%, and still more preferentially between 15 and 45%.

Preferably, the propellant is selected from dimethylether, C₃ to C₅ alkanes, such as n-butane, propane, isobutane, halogenated hydrocarbons, such as 1,1-difluoroethane, mixtures of dimethylether and C₃₋₅ alkanes, mixtures of 1,1-difluoroethane and dimethyl ether and/or C₃₋₅ alkanes.

More preferentially, the propellant is selected from dimethyl ether, C₃ to C₅ alkanes and their mixtures.

The composition according to the invention can comprise usual cosmetic additives selected notably from adhesive agents, reducing agents such as thiols, fatty substances, softeners, defoamers, filters, antiperspirants, acidifying agents, alkalinizing agents, dyes, pigments, perfumes, preservatives, anionic, cationic, nonionic or amphoteric surfactants, non-fixing and non-thickening polymers, silicones volatile or not, modified or not, soluble or insoluble, vegetable, animal, mineral or synthetic oils, perfumes, proteins and vitamins, glycerol and their mixtures.

The person skilled in the art will take care to select the possible additives and their quantity such that they do not prejudice the properties of the compositions of the present invention.

These additives are notably present in the composition according to the invention in a quantity ranging from 0 to 20% by weight in relation to the total weight of the composition.

The propellant is notably present in a quantity of between 2 and 90% by weight, preferably between 4 and 80%, better still between 5 and 65% by weight in relation to the total weight of the composition.

The compositions in accordance with the invention are packaged in an aerosol device usual in cosmetics.

The sprayed compositions can be in the form of a spray or a foam.

The compositions in accordance with the invention, sprayed from the aerosol device, can be used in rinsed or unrinsed application, as compositions for fixing and/or maintaining the hair, hair care compositions, shampoos, hair conditioning compositions, such as compositions designed to give the hair softness or even compositions for dressing the hair.

The present invention relates also to a cosmetic hair treatment process which consists of applying an effective quantity of a composition such as described above, to the hair and rinsing or not after a possible setting time.

According to a preferred embodiment of the invention, the composition, sprayed from the aerosol device, can be used as a non-rinsed hair-styling product.

In particular, the compositions in accordance with the invention are used to form and/or maintain the head of hair.

The following examples illustrate the present invention and should not be considered in any way as limiting the invention.

### EXAMPLES

Three hair-styling products were prepared from the following ingredients, the percentages being expressed by weight.

### Example 1

### Aerosol Foam

| | |
|---|---|
| Laponite XLG (Laporte) | 0.8% |
| Tego Betaine HS (Goldschmidt) | 0.4% |
| Viscophobe DB 1000 (Amerchol) | 0.86% |
| Preservatives | q.s. |
| AEROGAZ 3.2(1) | 6% |
| Water | q.s. for |
| | 100% |

(1) mixture of isobutane, propane and butane, marketed by ATOCHEM

### Example 2

### Aerosol Spray

| | |
|---|---|
| Laponite XLS (Laporte) | 0.3% |
| Ethanol | 20% |
| Water | 19.7% |
| DME | 60% |

### Example 3

### Aerosol Spray

| | |
|---|---|
| Laponite XLS (Laporte) | 0.4% |
| Luviset Si Pur (BASF) | 10% |
| Ethanol | 19% |
| Water | 10.6% |
| DME | 60% |

These products are applied preferably to wet or dry hair. After forming, a good hair-styling effect with no cosmetic disadvantages can be observed.

1. A cosmetic composition, notably for the hair, packaged in an aerosol device containing a propellant and a liquid phase which comprises, in a cosmetically acceptable medium:
(i) solid particles containing at least 10% by weight of at least one silicate, and
(ii) at least one polymer selected from fixing polymers, thickening polymers or a mixture thereof;
said solid particles containing less than 1% by weight of aluminum.

2. The composition as claimed in claim 1, characterized in that the silicate is selected from sodium, magnesium and/or lithium silicates.

3. The composition as claimed in either one of the preceding claims, characterized in that the solid particles have a number-average primary size of between 2 nm and 1 μm.

4. The composition as claimed in any one of the preceding claims, characterized in that the particles containing at least 10% by weight of at least one silicate have a number-average primary size of between 5 and 500 nm, and more preferentially between 10 and 250 nm.

5. The composition as claimed in any one of the preceding claims, characterized in that the particles contain at least 50% by weight of silicate, preferably 70% by weight, and more preferentially still, more than 90% by weight of silicate.

6. The composition as claimed in any one of the preceding claims, characterized in that the particles containing silicate are present in a quantity of between 0.01% and 10% by weight, and preferably between 0.5% and 3% by weight in relation to the total weight of the composition.

7. The composition as claimed in any one of the preceding claims, characterized in that the fixing polymers are selected from anionic and nonionic polymers.

8. The composition as claimed in claim 7, characterized in that the anionic polymer is selected from:
polymers comprising carboxylic units deriving from unsaturated carboxylic mono- or diacid monomers of formula: 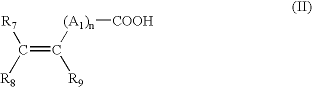
in which n is a whole number from 0 to 10, A denotes a methylene group, possibly linked to the carbon atom of the unsaturated group or to the neighboring methylene group when n is greater than 1 by means of a heteroatom such as oxygen or sulfur, R₇ denotes a hydrogen atom, a phenyl or benzyl group, R₈ denotes a hydrogen atom, a lower alkyl or carboxyl group, R9 denotes a hydrogen atom, a lower alkyl group, a —CH₂—COOH, phenyl or benzyl group;
polymers comprising units deriving from sulfonic acid such as vinylsulfonic, styrenesulfonic, acrylamidoalkylsulfonic units.

9. The composition as claimed in claim 7, characterized in that the nonionic polymer is selected from:
the homopolymers or copolymers of vinylpyrrolidone or vinylcaprolactam;
polyalkyloxazolines;
the homopolymers of vinyl acetate;
the copolymers of vinyl acetate and acrylic ester;
the copolymers of vinyl acetate and ethylene;
the copolymers of vinyl acetate and maleic ester;
the copolymers of polyethylene and maleic anhydride;
the homopolymers of alkyl acrylates and the homopolymers of alkyl methacrylates;
the copolymers of acrylic esters such as for example the copolymers of alkyl acrylates and alkyl methacrylates;
the copolymers of acrylonitrile and a nonionic monomer selected for example from butadiene and the alkyl (meth)acrylates;
the copolymers of alkyl acrylate and urethane; and
polyamides.

10. The composition as claimed in any one of claims 1 to 6, characterized in that the thickener is selected from copolymers of acrylic or methacrylic acid comprising at least one C1 to C30 alkyl acrylate unit and/or one urethane unit possibly substituted by a fatty chain.

11. The composition as claimed in any one of claims 1 to 6, characterized in that the thickener is selected from polymers or copolymers of acrylic and/or methacrylic acid, such as acrylic acid/ethyl acrylate copolymers and carboxyvinyl polymers.

12. The composition as claimed in any one of claims 1 to 6, characterized in that the fixing polymer is selected from:
derivatives of cationic cellulose;
guar gums containing cationic trialkylammonium groups.

13. The composition as claimed in any one of claims 1 to 6, characterized in that polymer (ii) is present in the cosmetic composition in a proportion of between 0.01 and 8%, and preferably of between 0.1 and 8% in relation to the total weight of the composition.

14. The composition as claimed in any one of the preceding claims, characterized in that the propellant is selected from dimethylether, C₃ to C₅ alkanes, such as n-butane, propane, isobutane, halogenated hydrocarbons, such as 1,1-difluoroethane, mixtures of dimethylether and C₃₋₅ alkanes, mixtures of 1,1-difluoroethane and dimethylether and/or C₃₋₅ alkanes.

15. The composition as claimed in the preceding claim, characterized in that the propellant is selected from dimethylether and C₃ to C₅ alkanes and their mixtures.

16. The composition as claimed in any one of the preceding claims, characterized in that the propellant is present in a quantity of between 2 and 90% by weight, preferably between 4 and 80%, better still between 5 and 65% by weight in relation to the total weight of the composition.

17. The composition as claimed in any one of the preceding claims, characterized in that it comprises usual cosmetic additives selected from adhesive agents, reducing agents such as thiols, fatty substances, softeners, defoamers, filters, antiperspirants, acidifying agents, alkalinizing agents, colorants, pigments, perfumes, preservatives, anionic, cationic, nonionic or amphoteric surfactants, non-fixing and non-thickening polymers, silicones volatile or not, modified or not, soluble or insoluble, vegetable, animal, mineral or synthetic oils, perfumes, proteins and vitamins, glycerol and their mixtures.

18. A cosmetic hair treatment process using a composition as claimed in any one of claims 1 to 17.

19. Use of a composition as claimed in any one of claims 1 to 17 in hair cosmetics, notably for forming and/or maintaining the head of hair.
